(19) United States
(12) Patent Application Publication
(10) Pub. No.: US 2014/0074133 A1
(43) Pub. Date: Mar. 13, 2014

(54) COMBINATION OF SHAVER AND OPTO-THERMAL MODIFICATION OF HAIR

(71) Applicant: PALOMAR MEDICAL TECHNOLOGIES, INC., Burlington, MA (US)

(72) Inventors: Gregory B. Altshuler, Lincoln, MA (US); James J. Childs, Bolton, MA (US); Ilya Yaroslavsky, North Andover, MA (US); Henry H. Zenzie, Dover, MA (US)

(73) Assignee: PALOMAR MEDICAL TECHNOLOGIES, INC., Burlington, MA (US)

(21) Appl. No.: 13/789,282

(22) Filed: Mar. 7, 2013

Related U.S. Application Data
(60) Provisional application No. 61/698,965, filed on Sep. 10, 2012.

(51) Int. Cl. A61N 5/00

(52) U.S. Cl. CPC A61N 5/00
USPC 606/167

(57) ABSTRACT

Methods, systems, and apparatus for hair treatment are disclosed which include applying treatment radiation to a skin treatment area and/or to one or more hairs to deposit energy in one or more hairs so as to modify the structure (e.g., the mechanical structure and/or the chemical structure of at least a portion of the hair(s)). The applied radiation can modify at least a portion of the hair (e.g., the hair tip) to make the hair less capable of re-entering the skin. Specifically, the proposed technique is directed to decreasing stiffness of at least portion of a hair through diminishing its flexural modulus as well as increasing the bend radius of its sharpest point. The methods and apparatus can treat and/or prevent pseudofolliculitis barbae (PFB) in the treatment area.

### RELATED APPLICATIONS

This application claims priority to U.S. Provisional Application Ser. No. 61/698,965 filed on Sep. 10, 2012, the contents of which are hereby incorporated by reference in their entirety.

### FIELD OF THE INVENTION

The present disclosure is directed to hair treatment methods, and specifically, to methods, systems, and apparatus for treatment and/or prevention of pseudofolliculitis barbae (PFB) utilizing treatment radiation (e.g., electromagnetic radiation or EMR), also referred to as optical radiation, which can include EMR, for example.

### BACKGROUND OF THE INVENTION

Pseudofolliculitis barbae (PFB) is a chronic papulopustular dermatitis of a bearded and/or shaved area resulting from reentry penetration of the epidermis by a growing hair. PFB occurs more prevalently in persons (males and females) having curly hair. Persons of darker (IV to VI) skin types are also particularly susceptible to this condition. Epidemiological studies (P K Perry et al. J. Am. Acad. Dermatol., 46:S113-S119, 2002) give estimates of incidence between 45% and 83% for black patients.

Pathogenesis of PFB is determined by a person's hair structure. The curved pattern of the hair growth is the principal characteristic that initiates the process. In persons having such a pattern of hair growth, the hair emerges from the skin surface and turns in the direction of the epidermis. The growth continues in a direction as if to complete a full circle (i.e., extrafollicular penetration), resulting in the hair penetrating into the skin. A foreign-body-type inflammatory reaction that follows produces a plurality of papules and, in a continuing spectrum, pustules. Alternatively, the emerging hair penetrates the wall of the follicle rather than arcing across a portion of skin prior to reentry (i.e., transfollicular penetration).

Conventional treatment approaches include 1) beard growing; 2) PFB-specific shaving techniques; 3) application of depilatories and topical creams (e.g., U.S. Pat. No. 6,352,690); and 4) electrolysis for treatment of ingrown hairs (e.g., U.S. Pat. No. 5,419,344).

Recently, laser-based treatment modalities, initially developed for removal of unwanted hair, have been applied for treatment of PFB. The conventional treatment modalities, however, suffer from a number of shortcomings. In particular, beard growing is not an option for many occupations and PFB-specific shaving techniques are cumbersome, time-consuming, and often not sufficiently effective. Topical depilatories can be difficult to use and may cause severe skin irritation, exacerbating the condition. Electrolysis can only be performed by a trained professional, is expensive and extremely time-consuming. Laser modalities can offer a curative solution to the problem; however, they may be sub-optimal for patients with darker skin types. Other light based treatments of PFB are disclosed by U.S. patent application Ser. No. 10/783,987 entitled Method and Apparatus for Treating Pseudofolliculitis Barbae and U.S. Pat. No. 7,044,959 entitled Method and Apparatus for Hair Growth Management, which are incorporated herein by reference in their entirety.

Thus, there exists a need in the art for a safe, effective, self-treatment method of PFB.

### SUMMARY

Methods, systems, and apparatus for hair treatment are disclosed which include applying treatment radiation to a skin treatment area and/or to one or more hairs to deposit energy in one or more hairs so as to modify the structure (e.g., the mechanical structure and/or the chemical structure and/or the geometrical structure of at least a portion of the hair(s)). The applied radiation can modify at least a portion of the hair (e.g., the hair tip) to make the hair less capable of re-entering the skin. Specifically, the proposed technique is directed to decreasing stiffness of at least portion of a hair through diminishing its flexural modulus as well as increasing the bend radius of its sharpest point. The disclosed methods, systems and apparatus can treat and/or prevent (PFB) in the treatment area.

In one embodiment, a razor (e.g., an electric razor) is combined with and/or integrated with a system for light based hair treatment to modify the hair structure to lessen and/or eliminate the incidence of extra follicular penetration and/or trans follicular penetration associated with PFB.

For example, in some aspects, a device is provided having one or more blades that are combined with an optical system that modifies the mechanical properties of a portion of the hair such as the tip (e.g., reduces the stiffness of the hair tip and/or makes the hair tip blunter). Changing the mechanical properties of the hair tip may or may not alter the geometry of the hair tip, what is necessary is that the hair tip be softened by changing and/or reducing its stiffness.

In accordance with various aspects of the present teachings, a device for hair modification is provided that includes a blade for cutting one or more hairs and a radiation source configured to provide treatment radiation to at least a portion of one or more hairs. In some embodiments, the device can additionally include a controller configured to provide treatment radiation to one or more cut hairs. In various aspects, the device can include a controller configured to provide treatment radiation solely to one or more cut hairs. In various aspects, the device can additionally include a contact sensor for determining the presence of one or more cut hairs.

These and other features of the applicants' teachings are set forth herein.

### BRIEF DESCRIPTION OF THE DRAWINGS

FIG. 1A illustrates an exemplary device in accordance with various aspects of the applicants' teachings.

FIG. 1B illustrates the exemplary device of FIG. 1A moving over the skin.

FIG. 1C, illustrates a portion of the exemplary device of FIG. 1A.

FIG. 2, illustrates another exemplary device in accordance with various aspects of the applicants' teachings.

FIG. 3 illustrates yet another exemplary embodiment in accordance with various aspects of the applicants' teachings.

FIG. 4 illustrates yet another exemplary embodiment in accordance with various aspects of the applicants' teachings.

FIG. 5 illustrates yet another exemplary embodiment in accordance with various aspects of the applicants' teachings.

FIG. 6 illustrates yet another exemplary embodiment in accordance with various aspects of the applicants' teachings.

### DETAILED DESCRIPTION

Certain exemplary embodiments will now be described to provide an overall understanding of the principles of the structure, function, and use of the systems and devices disclosed herein. One or more examples of these embodiments are illustrated in the accompanying drawings, which are not necessarily to scale. Those skilled in the art will appreciate that the systems and devices specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Methods, systems, and apparatus for hair treatment are provided herein which include applying treatment radiation to a skin treatment area and/or to one or more hairs so as to modify the structure (e.g., the mechanical structure and/or the chemical structure and/or the geometrical structure of at least a portion of the hair(s)). The applied radiation can modify at least a portion of the hair (e.g., the hair tip) to make the hair less capable of re-entering the skin. In various aspects, the methods, systems, and apparatus disclosed herein can treat and/or prevent (PFB) in the treatment area. In some aspects, one or more blades can be combined with a radiation source and/or an optical system to modify the mechanical properties of a portion of the hair such as the tip (e.g., to reduce the stiffness of the hair tip and/or make the hair tip blunter). By way of example, a razor (e.g., an electric razor) can be combined with and/or integrated with a system for light-based hair treatment to modify the hair structure to lessen and/or eliminate the incidence of extra-follicular penetration and/or trans-follicular penetration associated with PFB.

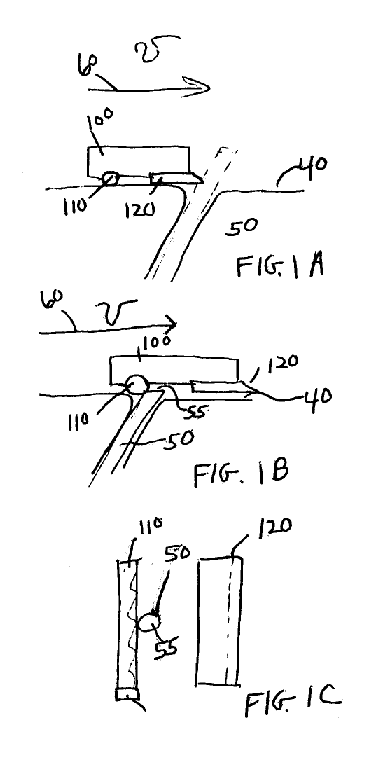

With reference now to FIGS. 1A-1C, an exemplary device 100 in accordance with various aspects of the present teachings is depicted in which a blade 120 of a razor (e.g., an electric razor) is integrated with a source of optical treatment radiation 110. The treatment radiation is optical radiation (e.g., EMR) having wavelength(s) in the range of about 200 to about 12,000 nm, about 300 to about 1500 nm, and, about 350 to about 450 nm. The source of the optical radiation can be, for example, a laser, an LED, or a lamp. The blade 120 can be substantially parallel with the source of optical treatment radiation 110. Referring to FIG. 1A, the device 100 can contact the surface of skin 40 in a region of hair growth. The device 100 is moved, while in contact with the surface of the skin 40, in the direction 60 such that the blade 120 of the razor cuts the hair 50. In some embodiments, the hair 50 is cut at a height that is level with the surface of the skin 40. In some embodiments, the hair 50 is cut at a height that is lower than the level with the surface of the skin 40. In other embodiments, the hair 50 is cut at a height that is higher than the level of the surface of the skin 40. After it is cut, the hair 50 has a newly cut tip 55. Referring now to FIGS. 1B and 1C, after the hair 50 is cut and while the device 100 continues to move in the direction 60 in some embodiments, the source of optical treatment radiation 110 contacts tip 55 of the hair 50 that sticks out of the follicle after being cut by the blade 120. Suitable sources of optical treatment radiation 110 may be, for example, a diode laser, a LED, and/or a lamp with or without a waveguide. The source of optical treatment radiation 110 provides optical radiation with sufficient energy density and power density to induce desired physical, chemical, and/or geometrical changes in the areas of the hair where the said radiation is absorbed through a photo thermal mechanism. The source of optical treatment radiation 110 is employed to modify the mechanical properties of the newly cut hair tip 55 (e.g., to soften and/or lessen the stiffness of the hair tip 55). The source of optical treatment radiation 110 may include an optical element that is being coupled from the source of energy to the hair tip 55. For example, the source of optical energy may be a diode laser coupled to a waveguide. For example, the optical treatment radiation is coupled to the hair tip 55 through direct contact between the source of optical treatment radiation 110 and the newly cut hair tip 55. The source of optical treatment radiation 110 may be provided to the hair tip 55, through, for example, a mechanism of disturbed total internal reflection (dTIR) resulting in the absorption of the optical radiation in hair in the area of direct contact with a waveguide (e.g., a sapphire waveguide).

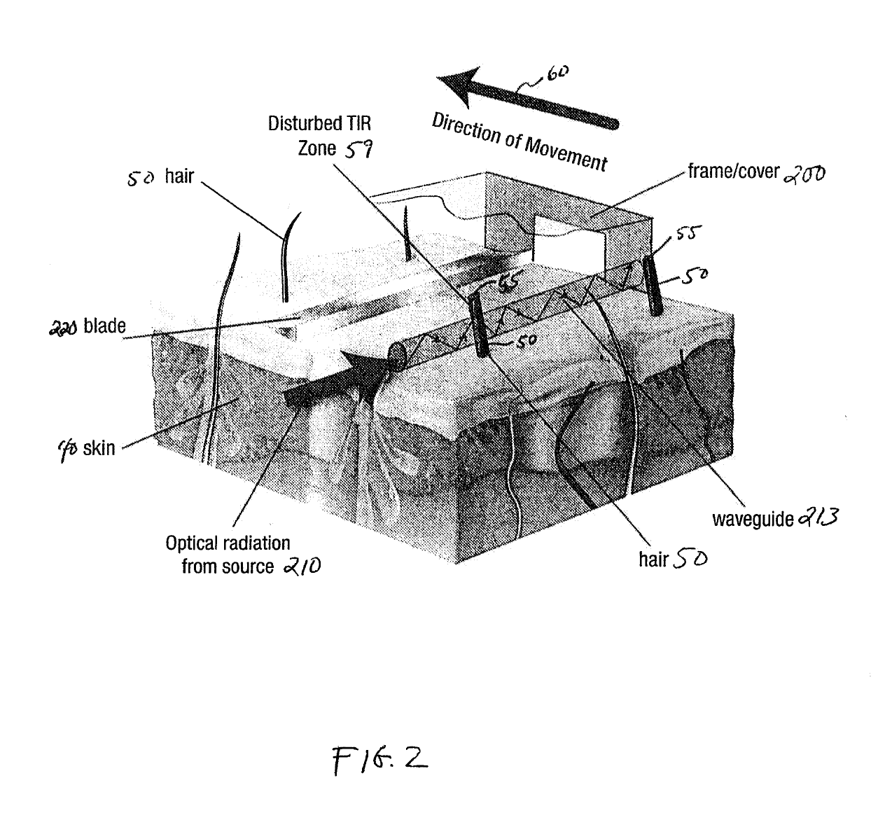

Optionally, reflective coupling may be provided through the blade 120 of the razor. FIG. 2 shows an exemplary device 200 for dTIR delivery having a frame that includes a blade 220 of a razor such as a hand-held razor (e.g., a manual razor or electric razor) that is integrated with a source of optical treatment radiation 210 and an optical element such as a waveguide 213. In one embodiment of the device 200 the blade 220 is substantially parallel with the source of optical treatment radiation 210 and/or the waveguide 213. The device 200 contacts the surface of skin 40 in a region of hair growth. The device 200 is moved, while in contact with the surface of the skin 40, in the direction 60 such that the blade 220 of the razor cuts the hair 50. In some embodiments, the hair 50 is cut at a height that is level with the surface of the skin 40. In other embodiments, the hair 50 is cut at a height that is lower than the level of the skin 40. In other embodiments, the hair 50 is cut at a height that is higher than the level of the surface of the skin 40. After the blade 220 cuts the hair 50 to form the newly cut tip 55, the optical radiation source 210 together with the waveguide 213 provide optical treatment radiation to modify (e.g., soften) the newly cut tip 55 (here, the tip is cut at a height that is higher than the level of the skin 40). The source of optical treatment radiation 210 provides optical radiation with sufficient energy density and power density to induce desired physical, chemical, and/or geometrical changes in the areas of the hair where the said radiation is absorbed through a photo thermal mechanism.

In some embodiments, the source of optical treatment radiation 210 together with the waveguide 213 provide treatment radiation to the cut tip 55 of the hair 50 when the device 200 is turned “on.” Alternatively, safety features may be built into the device 200 to ensure that the device 200 is in contact with the skin 40 surface. For example, in some embodiments, suitable contact sensors including, for example, a mechanical contact sensor, a light-gate sensor, an electrical (capacitive or impedance) sensor, or an optical sensor such as an image (camera) sensor are coupled to the device 200 to determine if the device 200 is in contact with the skin 40. Other suitable contact sensors are disclosed in U.S. Pat. No. 7,204,832, which is incorporated herein by reference in its entirety. A controller may be coupled to the device 200 and in response to the contact sensor the controller is configured to permit application of the treatment radiation from the source of optical treatment radiation 210 through the waveguide 213 when the contact sensor detects contact between the device 200 and skin 40.

Optionally, referring still to FIG. 2, the device 200, light source 210, and waveguide 213 work in concert to permit application of the treatment radiation from the source 210 only when the waveguide 213 is in physical contact with a hair 50. For example, the waveguide 213 may have a contact sensor that detects resistance when the waveguide 213 is pushed against the hair 50 (e.g., the recently cut hair tip 55); when resistance is detected, the optical radiation from the source 210 is signaled by the contact sensor to fire a treatment radiation suitable to treat the hair 50 (e.g., suitable to mechanically alter and/or soften the recently cut hair tip 55). The location where the waveguide 213 contacts and provides treatment radiation to the hair 50 creates a dTIR zone 59 in the hair 50 itself In this way, treatment radiation is absorbed by the hair 50 via contact with the waveguide 213

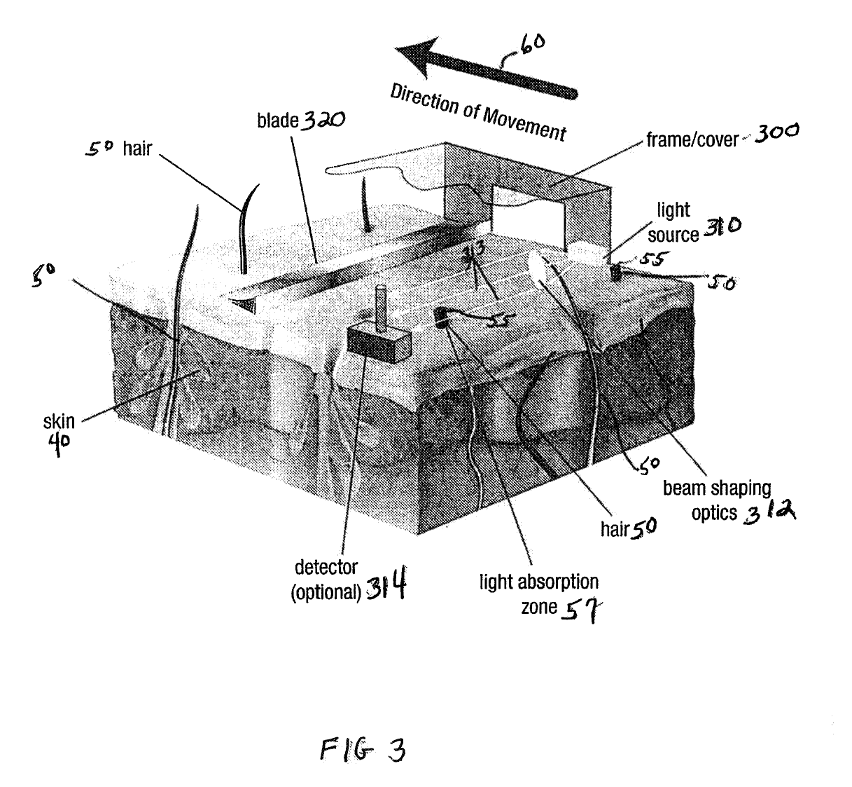

FIG. 3 shows another exemplary device 300 for direct beam delivery in accordance with various aspects of the present teachings. The device 300 includes a frame that includes a blade 320 of a razor (e.g., an electric razor) that is integrated with a source of optical treatment radiation 310. The source of optical treatment radiation 310 includes a light source that employs beam shaping optics 312 (e.g., a focusing lens). The device 300 includes a detector 314 for detecting the light output 313 that travels through the beam shaping optics 312 after having originated from the source of optical treatment radiation 310. In the device 300 the blade 320 is substantially parallel with the source of optical treatment radiation 310. The device 300 contacts the surface of skin 40 in a region of hair growth. The device 300 is moved, while in contact with the surface of the skin 40, in the direction 60 such that the blade 320 of the razor cuts the hair 50. In some embodiments, the hair 50 is cut at a height that is level with the surface of the skin 40. In other embodiments, the hair 50 is cut at a height that is lower than the level of the skin 40. In other embodiments, the hair 50 is cut at a height that is higher than the level of the surface of the skin 40. After the blade 320 cuts the hair 50 to form the newly cut tip 55 the light source 310 provides optical treatment radiation to modify (e.g., soften) the newly cut tip 55. The source of optical treatment radiation 310 provides optical radiation with sufficient energy density and power density to induce desired physical, chemical, and/or geometrical changes in the areas of the hair where the said radiation is absorbed through a photo thermal mechanism. In some embodiments, the source of optical treatment radiation 310 provides treatment radiation 313 when the device 300 is turned “on.”

Alternatively, safety features may be built into the device 300 to ensure that the device is in contact with the skin. For example, in some embodiments, suitable contact sensors including, for example, a mechanical contact sensor, a light-gate sensor, an electrical (capacitive or impedance) sensor, or an optical sensor such as an image (camera) sensor are coupled to the device 300 to determine if the device 300 is in contact with the skin 40. A controller is coupled to the device 300 and in response to the contact sensor when the contact sensor detects contact between the device 300 and skin 40 the controller is configured to permit application of the treatment radiation 313 that is focused through the beam shaping optics 312 after having exited the source of optical treatment radiation 310. Optionally, referring still to FIG. 3, the device 300 includes a detector 314 that works in concert with the light source 310 to permit application of the treatment radiation from the source 310 only when the detector 314 determines that a hair 50 is present in the path of the light output 313. For example, the light source 310 can provide a detection light emission (e.g., when it is determined that the device 300 is in contact with the skin 40) such that the detector 314 can determine, based on its analysis of the emission 313 received from the light source 310, that a hair 50 is in the path of the light output 313 from the beam shaping optics 312. Typically the determination of the presence of a hair 50 in the light output 313 is based on the melanin content of the hair. Once the detector 314 determines that hair is present in the path of the light output 313 the treatment radiation is permitted to be illuminated from the source of optical treatment radiation 310. For example, a controller coupled to the device is configured to permit application of the treatment radiation from the source of optical treatment radiation 310 once the detector 314 determines that hair is present in the path of the light output 313. In this way, the newly cut tip 55 can be modified by the device 300.

Still referring to FIG. 3, optionally, the light source 310 provides a level of radiation (e.g., a diagnostic level of radiation) and when the detector 314 detects a drop in the level of radiation (e.g., in the level of the diagnostic radiation) then the device 300 determines that hair 50 is present in the path of the light output 313 due to absorption of light by the hair 50 in the light absorption zone 57. Accordingly, the light source 310 increases the level of radiation to achieve the desired treatment of the newly cut hair tip 55 and/or of the hair 50.

Optionally, one could use short wavelengths with high absorption to determine the presence of melanin.

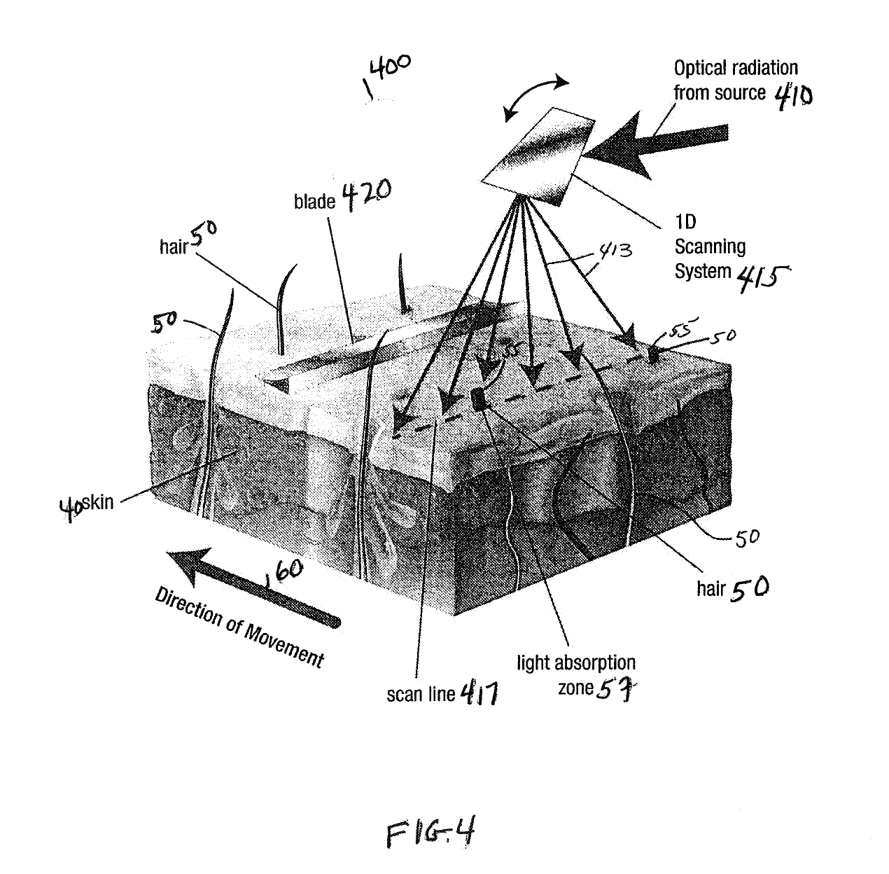

FIG. 4 shows an exemplary device 400 for scanned beam delivery in accordance with various aspect of the applicants' present teachings. The device 400 includes a blade 420 of a razor (e.g., an electric razor) that is integrated with a source of optical treatment radiation 410. The source of optical treatment radiation 410 includes a light source that employs a 1D scanning system (e.g., a mirror). All or a portion of the device 400 contacts the surface of skin 40 in a region of hair growth. The device 400 is moved, while in contact with the surface of the skin 40, in the direction 60 such that the blade 420 of the razor cuts the hair 50. In some embodiments, the hair 50 is cut at a height that is level with the surface of the skin 40. In other embodiments, the hair 50 is cut at a height that is lower than the level of the skin 40. In other embodiments, the hair 50 is cut at a height that is higher than the level of the surface of the skin 40. After the blade 420 cuts the hair 50 to form the newly cut tip 55, the optical radiation source 410 provides optical treatment radiation 413 that is scanned via a 1D scanning system 415 that scans optical treatment radiation 413 over the surface of the skin 40 (in the path of the scan line 417) to modify (e.g., soften) the newly cut tip 55. In some embodiments, the optical radiation source 410 is focused to provide a spot size that is about the size of a hair e.g., about 100 microns, from about 10 microns to about 200 microns, or from about 50 microns to about 150 microns. In some embodiments, the 1D scanning systems 415 provides focusing (e.g., is a focusing mirror). The source of optical treatment radiation 410 provides optical radiation with sufficient energy density and power density to induce desired physical, chemical, and/or geometrical changes in the areas of the hair where the said radiation is absorbed through a photo thermal mechanism. In some embodiments, the source of optical treatment radiation 410 provides treatment radiation that is scanned via a 1D scanning system 415 when the device 400 is turned “on.”

In some embodiments, a controller is coupled to the device 400 and in response to a contact sensor the controller is configured to permit scanned application of the treatment radiation 413 from the source of optical treatment radiation 410 when the contact sensor detects contact between the device 400 and skin.

Optionally, the scanned beam 413 that travels through the 1D scanning system 415 is a free beam.

In some embodiments, the 1D scanning system 415 features feedback control to provide feedback control detection such that a detection radiation is the scanned beam 413 that is scanned by the 1D scanning system 415 and when the feedback control detects the presence of hair it prompts the optical radiation source 410 to provide optical treatment radiation 410 that is fired at the hair tip 55. Suitable feedback control mechanisms can include an array such as a CCD camera that detects the presence of hair on the surface of the skin. The scanned treatment radiation 413 may be controlled such that the optical treatment radiation hits the target hair 55; this intersection may be referred to as the light absorption zone 57.

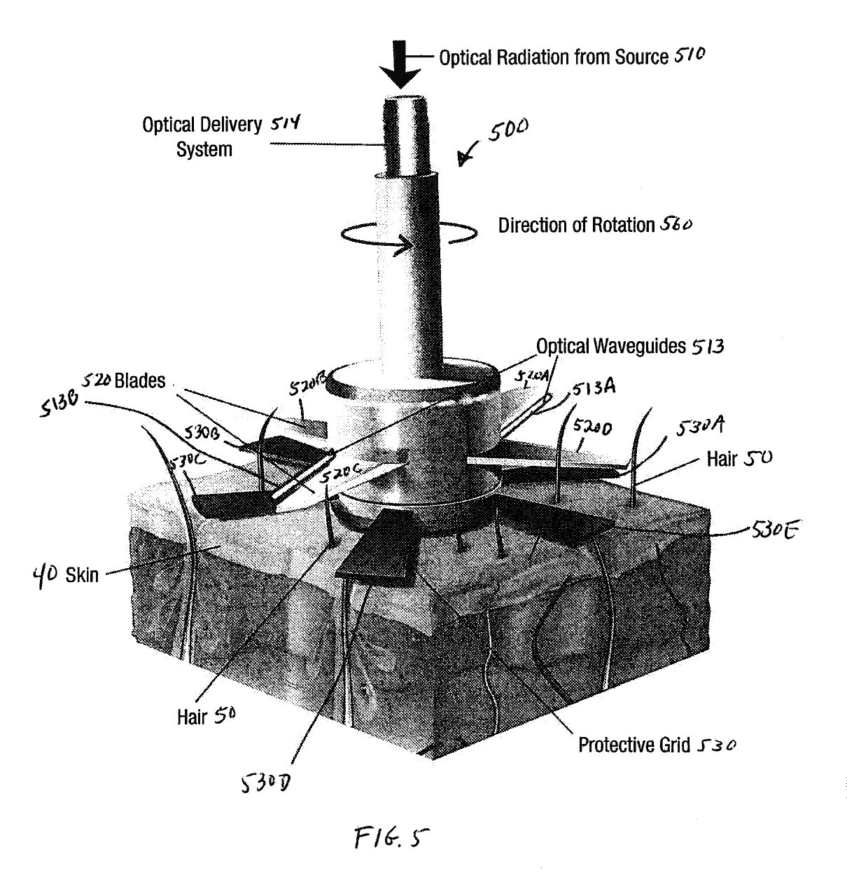

FIG. 5 shows another exemplary device 500 for disturbed TIR delivery that includes a Rotary Shaver in accordance with various aspects of the present teachings. The device 500 includes one or more blades 520 (e.g., blades 520A, 520B, 520C, and 520D) of a razor (e.g., an electric razor) that are integrated with a source of optical treatment radiation 510, an optical delivery system 514 and one or more optical waveguides 513 (e.g., waveguides 513A and 513B). The device 500 contacts the surface of skin 40 in a region of hair growth. The device 500 includes a protective grid 530 comprised of one or more protective grid members (530A, 530B, 530C, 530D, 530E etc.) and the protective grid 530 makes the skin in contact therewith immobile to ensure that the skin 40 avoids contact with the blades 530 (e.g., during use of the device 500 on someone's skin). At least a portion of the device 500 moves in the direction of rotation 560 such that the blades 520 of the razor (e.g., blades 520A, 520B, 520C, and 520D) move in rotation direction 560 to cut the hair(s) 50 in their path. In some embodiments, the hair 50 is cut at a height that is level with the surface of the skin 40. In other embodiments, the hair 50 is cut at a height that is lower than the level of the skin 40. In other embodiments, the hair 50 is cut at a height that is higher than the level of the surface of the skin 40. After the blades 520 cut the hair(s) 50 to form the newly cut tip, the optical radiation source 510 together with the optical delivery system 514 and waveguide(s) 513 (e.g., 513A and 513B) provide optical treatment radiation to modify (e.g., soften) the newly cut hair tip(s). The optical delivery system 514 can be, for example, an open beam, a fiber, and/or a waveguide. The source of optical treatment radiation 510 provides optical radiation with sufficient energy density and power density to induce desired physical, chemical, and/or geometrical changes in the areas of the hair where the said radiation is absorbed through photo thermal mechanism.

In some embodiments, the source of optical treatment radiation 510 together with the waveguide(s) 513 provide treatment radiation to the cut tip(s) of the hair(s) 50 when the device 500 is turned “on.” Alternatively, safety features may be built into the device 500 to ensure that the device is in contact with the skin. For example, in some embodiments, suitable contact sensors including, for example, a mechanical contact sensor, a light-gate sensor, an electrical (capacitive or impedance) sensor, or an optical sensor such as an image (camera) sensor are coupled to the device 500 to determine if the device 500 is in contact with the skin 40. A controller is coupled to the device 500 and in response to the contact sensor the controller is configured to permit application of the treatment radiation from the source of optical treatment radiation 510 through the optical delivery system 514 and then through the waveguide(s) 513 (e.g., waveguides 513A and 513B) when the contact sensor detects contact between the device 500 and the skin 40.

Optionally, referring still to FIG. 5, the device 500, light source 510, optical delivery system 514 and waveguide(s) 513 work in concert to permit application of the treatment radiation from the source 510 only when at least one of the waveguide(s) 513 (e.g., 513A or 513B) is in contact with the hair 50 (e.g., when waveguide 513B is in contact with the newly cut tip of a hair 50 cut by blade 520C after the device 500 turns in the direction of rotation 560 to cut the hair 50). For example, each of the waveguide(s) 513 (e.g., waveguides 513A and 513B) may have a contact sensor that detects resistance when the individual waveguide 513 is pushed against a hair 50 or a portion of a hair 50 (e.g., a recently cut hair tip); when resistance is detected the optical radiation from the source 510 is signaled by the contact sensor to fire a treatment radiation suitable to treat the hair 50 (e.g., suitable to mechanically alter and/or soften the recently cut hair tip). The location where the waveguide 513 contacts the hair 50 creates a disturbed TIR (total internal reflection) zone in the recently cut hair itself. In this way, treatment radiation is absorbed by the hair 50 via contact with the waveguide(s) 513. More specifically, a hair 50 is cut by blade 520C and thereafter waveguide 513B contacts the recently cut hair 50 (e.g., the recently cut hair tip) and the treatment radiation is absorbed by the cut hair via contact with the waveguide 513B.

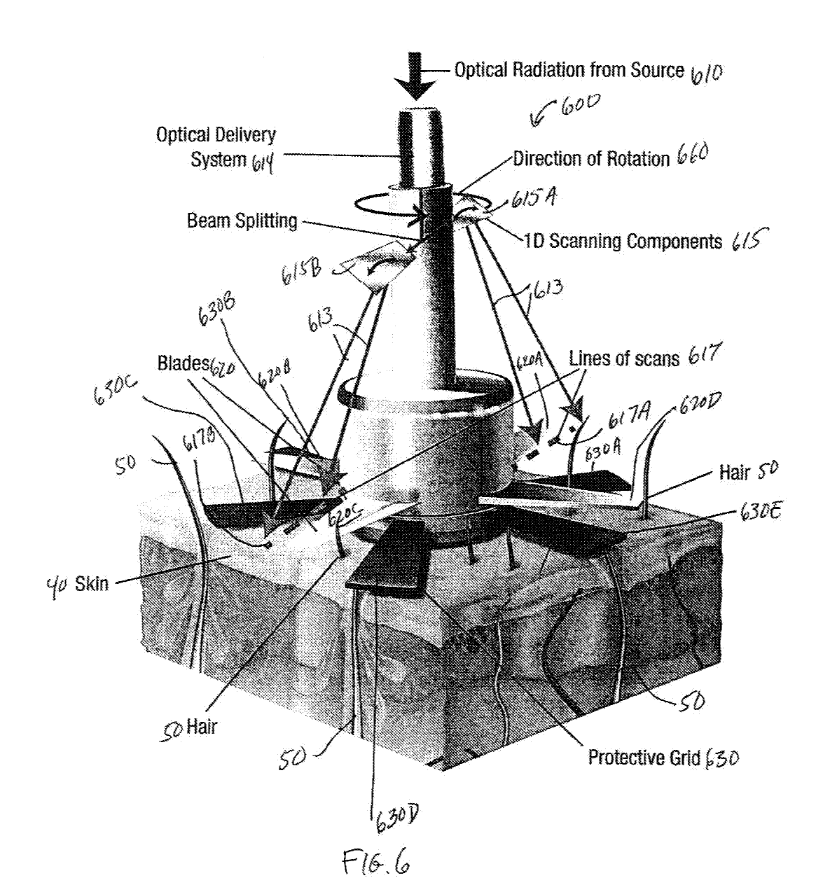

FIG. 6 shows an exemplary device 600 for scanned beam delivery that includes a Rotary Shaver. The device 600 includes one or more blades 620 (e.g., blades 620A, 620B, 620C, and 620D) of a razor (e.g., an electric razor) that are integrated with one or more sources of optical treatment radiation 610. The device 600 contacts the surface of skin 40 in a region of hair growth. The device 600 includes a protective grid 630 comprised of one or more protective grid members (630A, 630B, 630C, 630D, 630E etc.) and the protective grid 630 makes the skin in contact therewith immobile to ensure that the skin 40 avoids contact with the blades 630. The device 600 moves in the direction of rotation 660 such that the blades 620 of the razor (e.g., blades 620A, 620B, 620C, and 620D) move in rotation direction 660 to cut the hair(s) 50 in their path. In some embodiments, the hair 50 is cut at a height that is level with the surface of the skin 40. In other embodiments, the hair 50 is cut at a height that is lower than the level of the skin 40. In other embodiments, the hair 50 is cut at a height that is higher than the level of the surface of the skin 40. After the blades 620 cut the hair(s) 50 to form the newly cut tip, the optical radiation source 610 provides optical radiation that is delivered via scanning components (e.g., 1D scanning components 615A and 615B). FIG. 6 depicts optical radiation from the source 610 being delivered via an optical delivery system 614 (e.g., a waveguide or fiber) that exits the optical delivery system 614 as a split beam 613 to be scanned by the 1D scanning components 615A and 615B.

The optical treatment radiation 613 is scanned via the 1D scanning system 615A and 615B to scan optical treatment radiation 613 over the surface of the skin 40 (in the path of the scan lines 617A and 617B) to modify (e.g., soften) the newly cut tip(s). In some embodiments, the optical radiation source 610 is focused to provide a spot size that is about the size of a hair e.g., about 100 microns, or from about 10 microns to about 200 microns, or from about 50 microns to about 150 microns. In some embodiments, the 1D scanning systems 615A and 615B provides focusing (e.g., is a focusing mirror). The source of optical treatment radiation 610 provides optical radiation with sufficient energy density and power density to induce desired physical, chemical, and/or geometrical changes in the areas of the hair where the said radiation is absorbed through photo thermal mechanism. In some embodiments, the source of optical treatment radiation 610 provides treatment radiation 613 that is scanned via a 1D scanning system(s) 615A and 615B when the device 600 is turned “on.”

In some embodiments, a controller is coupled to the device 600 and in response to a contact sensor the controller is configured to permit scanned application of the treatment radiation 613 from the source of optical treatment radiation 610 when the contact sensor detects contact between the device 600 and skin 40.

Optionally, the scanned beam 613 that travels through the 1D scanning system 615A and 615B is a free beam (not shown).

In some embodiments, the 1D scanning system 615 features feedback control to provide feedback control detection such that a detection radiation is the scanned beam 613 that is scanned by the 1D scanning system 615A and 615B and when the feedback control detects the presence of hair it prompts the optical radiation source 610 to provide optical treatment radiation 613 that is fired at the hair 50 (e.g., at the hair tip). Suitable feedback control mechanisms can include an array such as a CCD camera that detects the presence of hair 50 on the surface of the skin. The scanned treatment radiation 613 may be controlled such that the optical treatment radiation hits the target hair 50.

In any of the disclosed embodiments, the hair 50 to be cut may be pre-heated (e.g., pre-heated via light energy such as EMR) and the blade used to cut the hair 50 may be warm or may be cold before the final cut of the hair. It may be desirable to pre-heat the hair 50 at about the height of the hair that will actually be cut accounting for the blade pulling the hair up slightly. By employing heat to heat the hair, the hair to be cut is softened hair and after it is cut it will be short and soft. Warm hair will be relatively easier to cut than cold hair.

In any of the disclosed embodiments, a linear lamp may be employed together with a focusing device.

What is claimed is:

1. A device for hair modification, comprising:
a blade to cut one or more hairs; and
a radiation source configured to provide treatment radiation to at least a portion of one or more hairs.

2. The device of claim 1 further comprising a controller configured to provide treatment radiation to one or more cut hairs.

3. The device of claim 1 further comprising a controller configured to provide treatment radiation solely to one or more cut hairs.

4. The device of claim 1 further comprising a contact sensor for determining the presence of one or more cut hairs.
